(19) United States
(12) Patent Application Publication
(10) Pub. No.: US 2020/0275909 A1
(43) Pub. Date: Sep. 3, 2020

(54) CONNECTORS FOR PATIENT INTERFACE MODULE AND ULTRASOUND IMAGING DEVICE

(71) Applicant: KONINKLIJKE PHILIPS N.V., EINDHOVEN (NL)

(72) Inventors: James R. MNIECE, WALTHAM, MA (US); Joe D'Anello, MEDFORD, MA (US); Norman HOSSACK, FOLSOM, CA (US)

(21) Appl. No.: 16/645,848

(22) PCT Filed: Sep. 7, 2018
(86) PCT No.: PCT/EP2018/074233

§ 371 Date: Mar. 10, 2020

Related U.S. Application Data
(60) Provisional application No. 62/557,270, filed on Sep. 12, 2017.

(51) Int. Cl. A61B 8/00; A61B 8/12; A61B 8/08

(52) U.S. Cl. CPC A61B 8/4433; A61B 8/12; A61B 8/461; A61B 8/5207; A61B 8/0883

(57) ABSTRACT

System, devices, and methods for ultrasound imaging are provided, which may include a patient interface module (PIM) positioned between an ultrasound imaging device and a console. The PIM may include a proximal connector configured to interface with the console, a distal connector configured to interface with a connector of the ultrasound imaging device, and a cable extending between the proximal connector and the distal connector. The proximal connector may include a plurality of transformers.

### RELATED APPLICATION

This application claims the benefit of and priority to U.S. Provisional Ser. No. 62/557,270, filed Sep. 12, 2017, which is incorporated by reference herein.

### TECHNICAL FIELD

The present disclosure relates generally to ultrasound imaging devices.

### BACKGROUND

Diagnostic and therapeutic ultrasound catheters have been designed for use inside many areas of the human body. In the cardiovascular system, a common diagnostic ultrasound methods is intraluminal ultrasound imaging with intra-cardiac echocardiography (ICE) being a specific example of intraluminal imaging. Typically a single rotating transducer or an array of transducer elements is used to transmit ultrasound at the tips of the catheters. The same transducers (or separate transducers) are used to receive echoes from the tissue. A signal generated from the echoes is transferred to a console which allows for the processing, storing, display, or manipulation of the ultrasound-related data.

Intraluminal imaging catheters such as ICE catheters (e.g., Siemens Acunav, St. Jude ViewFlex) are generally used to image heart and surrounding structures, for example, to guide and facilitate medical procedures, such as transseptal lumen punctures, left atrial appendage closures, atrial fibrillation ablation, and valve repairs. Commercially-available ICE catheters have distal ends which can be articulated by a steering mechanism located in a handle at the proximal end of the catheter. For example, an intraluminal imaging catheter such as an ICE catheter may be inserted through the femoral or jugular vein when accessing the anatomy, and steered in the heart to acquire images necessary to the safety of the medical procedures.

An ICE catheter typically includes imaging transducers for ultrasound imaging that generates and receives acoustic energy. The imaging core may include a lined array of transducer elements or transducer elements arranged in any suitable configuration. The imaging core is encased in an imaging assembly located at a furthest distal tip of the catheter. The imaging assembly is covered with acoustic adhesive materials. An electrical cable is soldered to the imaging core and extends through the core of the body of the catheter. The electrical cable may carry control core signals and echo signals to facilitate imaging of the heart anatomy.

ICE catheters utilize electronics in the tip, which is manipulated inside the heart for imaging. To operate the electronics, there are signal lines that connect the electronics at the tip to an external console. To ensure the device does not pass excess leakage current from the console, through the catheter, and to the patient, electrical isolation networks are used to eliminate the risk of sending the patient into cardiac arrest. The components required for these isolation networks tend to be heavy and large in size. They also require electromagnetic interference (EMI) protection (components must be surrounded by a metal cage). The bulk and weight of these components requires large and heavy interface components used with ICE catheters. These components may be unwieldy and may ultimately require placement or mounting on the surgical bed during operation. This can be intrusive and is unappealing visually. Thus, needs exist for interface components with a reduced size and weight that do not include isolation circuitry.

### SUMMARY

An ultrasound imaging system is provided by the present disclosure. The ultrasound imaging system can include a patient interface module (PIM) communicatively positioned between the intra-cardiac echocardiography (ICE) catheter and a console or control/processing system configured to receive and display imaging information from the imaging device. The PIM includes a proximal connector, a distal connector, and a cable extending between the connectors. The proximal connector of the PIM couples to the console, while the distal connector of PIM couples to a proximal connector of the ICE catheter. The PIM, PIM connectors, and the ICE catheter connector are advantageously smaller, lighter, and more easily to handle in clinical environment.

Technical advancements described herein include a PIM with reduced size and weight that is operable to support two- and three-dimensional imaging catheters. Furthermore, a PIM is provided that does not require a separate isolation box. For example, isolation components may be integrated directly into a PIM connector. A connector that connects the PIM and a catheter is also provided that does not require isolation components.

An ultrasound imaging system is provided by the present disclosure, which may include: a patient interface module communicatively positioned between an ultrasound imaging device and a console, the patient interface module comprising: a proximal connector configured to interface with the console; a distal connector configured to interface with a connector of the ultrasound imaging device; and a cable extending between the proximal connector and the distal connector, wherein the proximal connector comprises a plurality of transformers.

In some embodiments, the system further comprises the ultrasound imaging device. The ultrasound imaging device may include an intra-cardiac echocardiography (ICE) catheter. The ultrasound imaging device may be configured to output signals via a plurality of channels, wherein a quantity of the plurality of transformers equals a quantity of the plurality of channels. The system may further include the console, wherein the console may be configured to process the signals output by the ultrasound imaging device and display an ultrasound image based on the processed signals.

In some embodiments, the distal connector has a width of approximately 2 inches and a length of approximately 4.75 inches. In some embodiments, the proximal connector has a width of approximately 3 inches and a length of approximately 4.36 inches. In some embodiments, the distal connector has a weight of approximately 0.2 lbs. and the proximal connector has a weight of approximately 0.6 lbs. The distal connector may include one or more electronic components. The one or more electronic components may include a memory configured to facilitate communications between the ultrasound imaging device and the console. The one or more electronic components may include a plurality of integrated circuits configured to increase signal integrity. The one or more electronic components may include a plurality of amplifiers configured to strengthen signals output by the ultrasound imaging device.

An ultrasound imaging system is provided by the present disclosure, which may include: an ultrasound imaging device in communication with a console, comprising: a flexible elongate member comprising a proximal portion and a distal portion; an ultrasound transducer at the distal portion; a plurality of conductive wires extending along the flexible elongate member; and a connector at the proximal portion, wherein the connector comprises: a printed circuit board assembly (PCBA) configured to transmit imaging data obtained by the ultrasound transducer.

In some embodiments, the plurality of conductive wires extends from the ultrasound transducer to the connector and provides an electrical connection between the ultrasound transducer and the connector. The connector may include a first substrate at which the plurality of conductive wires terminates and a second substrate in communication with the first substrate, wherein the PCBA is disposed on the second substrate. The first substrate may be attached to a coupling module on the second substrate.

In some embodiments, the proximal connector has a width of approximately 3 inches and a length of approximately 4.36 inches. In some embodiments, the first substrate has a width of approximately 0.01 inch and a length of approximately 0.5 inch. Each conductive wire may serve as a data channel to transmit data from the ultrasound transducer to the connector. In some embodiments, the imaging data is stored in an EEPROM within the PCBA.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging system according to embodiments of the present disclosure.

FIG. 2 is a perspective view of an imaging assembly according to embodiments of the present disclosure.

FIG. 3 is a top view of a tip member according to embodiments of the present disclosure.

FIG. 4 is a schematic diagram illustrating the beam-forming of an intraluminal imaging device according to embodiments of the present disclosure.

FIG. 5 is a schematic diagram illustrating aspects of an intraluminal imaging device according to embodiments of the present disclosure.

FIG. 6 is a flow diagram of a method of performing intraluminal imaging with an intraluminal device according to aspects of the disclosure.

FIG. 7 is a schematic diagram of an ultrasound imaging system according to embodiments of the present disclosure.

FIG. 8A is an illustration of a patient interface module (PIM) according to embodiments of the present disclosure.

FIG. 8B is a perspective view of a proximal connector of the PIM of FIG. 8A according to embodiments of the present disclosure.

FIG. 9A is a perspective view of a connector assembly including a proximal connector of an ultrasound imaging device and a distal connector of the PIM of FIG. 8A according to embodiments of the present disclosure.

FIG. 9B is a perspective view illustrating components of the proximal connector of the ultrasound imaging device and the distal connector of the PIM of FIG. 9A according to embodiments of the present disclosure.

FIG. 10 is a perspective view of a proximal connector of an ultrasound imaging device according to embodiments of the present disclosure.

FIG. 11 is a perspective view of a connector substrate according to embodiments of the present disclosure.

FIG. 12 is another perspective view of a connector substrate according to embodiments of the present disclosure.

FIG. 13 is a perspective view of a prior art PIM.

FIG. 14 illustrates the prior art connecter interface and a connector assembly according to embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the ICE system is described in terms of intraluminal imaging, it is understood that it is not intended to be limited to this application. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

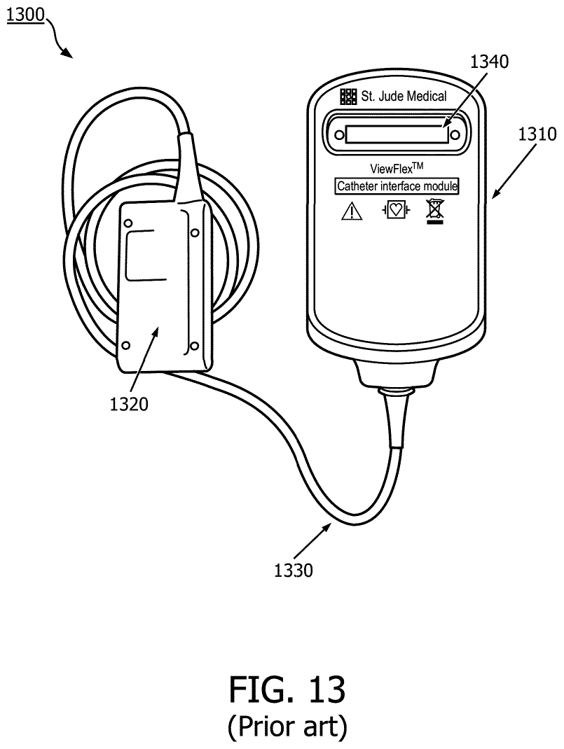

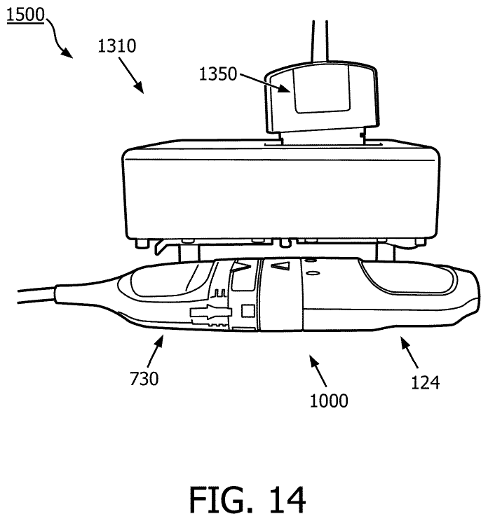

To illustrate some of the advantages of the present disclosure, FIG. 13 illustrates an existing patient interface module (PIM) 1300. This PIM 1300 includes large and heavy electronic components such as a larger power converter 1320, an isolation box 1310, and a cable 1330 between the power converter and converter box. In some embodiments, the isolation box alone may weigh over 2 lbs, and the combined weight of the cable assembly may be over 3 lbs. The isolation box 1310 typically includes a number of bulky components, including an array of transformers, and a large port 1340. For example, when the ICE transducer transmits imaging data via 64 channels, an isolation box along the cable between the ICE catheter and the signal processing system can include 64 transducers. The large port 1340 is configured to receive a large connector 1350 as shown in FIG. 14. Accordingly, existing connectors include large components and are not easily removable or accessible. Because the size and weight of these components, they can be difficult to maneuver in the clinical environment and take up valuable space in a procedure.

The embodiments of the present disclosure provide for a PIM with reduced weight and size that supports two- and three-dimensional imaging catheters. One advantage is that the PIM of the present disclosure may not require a separate isolation box (such as isolation box 1310) as is required by the existing PIM 1300. Furthermore, connectors between the PIM and a catheter are not required to include isolation circuitry. Instead, isolation circuitry may be included in one or more connectors between the PIM and processing systems.

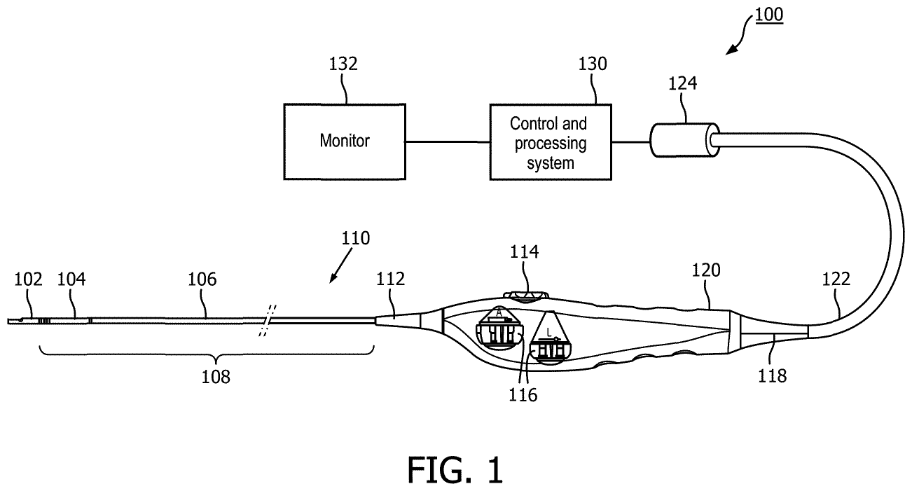

FIG. 1 is a schematic diagram of an intraluminal imaging system 100 according to embodiments of the present disclosure. The system 100 may include an ultrasound imaging device 110, a connector 124, a control and processing system 130 (for example, a console and a computer), and a monitor 132. The ultrasound imaging device 110 includes an imaging assembly 102 at the tip of a flexible elongate member 108, and a handle 120. The flexible elongate member 108 includes a distal portion 104 and a proximal portion 106. The distal end of the distal portion 104 is attached to the imaging assembly 102. The proximal end of the proximal portion 106 is attached to the handle 120, for example, by a resilient strain reliever 112. The handle 120 may be used for manipulation of the ultrasound imaging device 110 and manual control of the ultrasound imaging device 110. The imaging assembly 102 can include an imaging core with ultrasound transducer elements and associated circuitry. The handle 120 can include actuators 116, a clutch 114, and other steering control components for steering the ultrasound imaging device 110. The steering may include deflecting the imaging assembly 102 and the distal portion 104, as described in greater details herein.

The handle 120 is connected to the connector 124 via another strain reliever 118 and a connection cable 122. The connector 124 may be configured to provide suitable configurations for interconnecting the control and processing system 130 and the monitor 132 to the imaging assembly 102. The control and processing system 130 may be used for processing, storing, analyzing, and manipulating data, and the monitor 132 may be used for displaying obtained signals generated by the imaging assembly 102. The control and processing system 130 can include one or more processors, memory, one or more input devices, such as keyboards and any suitable command control interface device. The control and processing system 130 can be operable to facilitate the features of the intraluminal imaging system 100 described herein. For example, a processor can execute computer readable instructions stored on the non-transitory tangible computer readable medium. The monitor 132 can be any suitable display device, such as liquid-crystal display (LCD) panel or the like.

In operation, a physician or a clinician may advance the flexible elongate member 108 into a vessel within a heart anatomy. By controlling the actuators 116 and the clutch 114 on the handle 120, the physician or clinician can steer the flexible elongate member 108 to a position near the area of interest to be imaged. For example, one actuator 116 may deflect the imaging assembly 102 and the distal portion 104 in a left-right plane and the other actuator 116 may deflect the imaging assembly 102 and the distal portion 104 in an anterior-posterior plane, as discussed in greater details herein. The clutch 114 provides a locking mechanism to lock the positions of the actuators 116 and in effect lock the deflection of the flexible elongate member while imaging the area of interest.

The imaging process may include activating the ultrasound transducer elements on the imaging assembly 102 to produce ultrasonic energy. A portion of the ultrasonic energy is reflected by the area of interest and the surrounding anatomy, and the ultrasound echo signals are received by the ultrasound transducer elements. The connector 124 transfers the received echo signals to the control and processing system 130 where the ultrasound image is reconstructed and displayed on the monitor 132. In some embodiments, the processing system 130 can control the activation of the ultrasound transducer elements and the reception of the echo signals. In some embodiments, the control and processing system 130 and the monitor 132 may be part of a same system.

The system 100 may be utilized in a variety of applications such as transseptal punctures, left atrial appendage closures, atrial fibrillation ablation, and valve repairs and can be used to image vessels and structures within a living body. Although the system 100 is described in the context of intraluminal imaging procedures, the system 100 is suitable for use with any catheterization procedure, e.g., ICE. In addition, the imaging assembly 102 may include any suitable physiological sensor or component for diagnostic, treatment, and/or therapy. For example, the imaging assembly can include an imaging component, an ablation component, a cutting component, a morcellation component, a pressure-sensing component, a flow-sensing component, a temperature-sensing component, and/or combinations thereof.

In some embodiment, the ultrasound imaging device 110 includes a flexible elongate member 108 that can be positioned within a vessel. The flexible elongate member 108 may have a distal portion 104 and a proximal portion 106. The ultrasound imaging device 110 includes an imaging assembly 102 that is mounted within the distal portion 104 of the flexible elongate member 108.

In some embodiments, the intraluminal imaging system 100 is used for generating two-dimensional and three-dimensional images. In some examples, the intraluminal imaging system 100 is used for generating X-plane images at two different viewing directions perpendicular to each other.

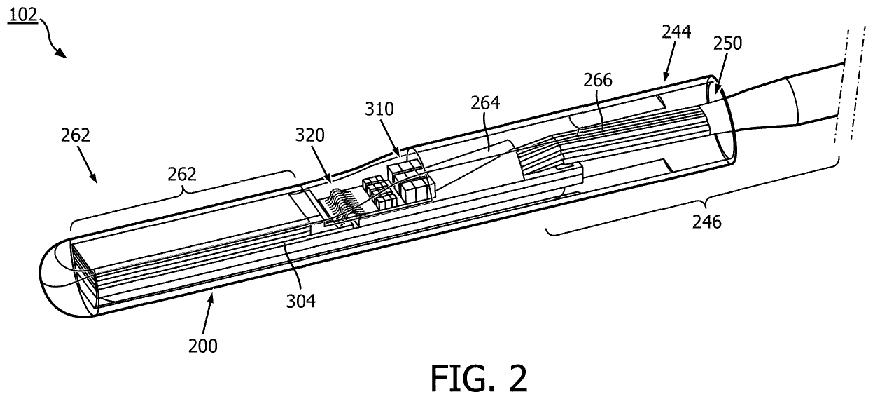

FIG. 2 is a perspective view of the imaging assembly 102 described above with respect to FIG. 1. The imaging assembly 102 may include the imaging core 262 that is positioned within a tip member 200. The imaging core 262 is coupled to an electrical cable 266 via an electrical interconnection 264. The electrical cable 266 extends through the alignment portion 244 and the interface portion 246 of the inner cavity 250. The electrical cable 266 can further extend through the flexible elongate member 108 as shown in FIG. 1.

The configuration and structure of the tip member 200 described above provide several benefits. The benefits include providing safe and easy delivery of the catheter, providing improved tensile strength for steering and navigation, providing consistent alignment, and providing improved image quality. For example, the outer geometry of the tip member 200 is configured to provide smooth surfaces and smooth edges with small radii. The smooth edges reduce friction when the tip member 200 traverses a vessel during insertion. The smooth surfaces prevent tears and/or damages to tissue structures during the insertion. In addition, the smooth edges and smooth surfaces can facilitate crossing of a septum or other anatomical feature during a catheterization procedure. In some embodiments, the material type and the wall thickness of the tip member 200 are selected to minimize acoustic distortion, attenuation, and/or reflection. The internal geometry of the tip member 200 is configured to facilitate alignment during manufacturing. The tip member 200 can also include other features, for example, a guidewire lumen, one or more holes, or other geometry to accommodate additional devices or features such as pressure sensors, drug delivery mechanisms, and/or any suitable interventional features.

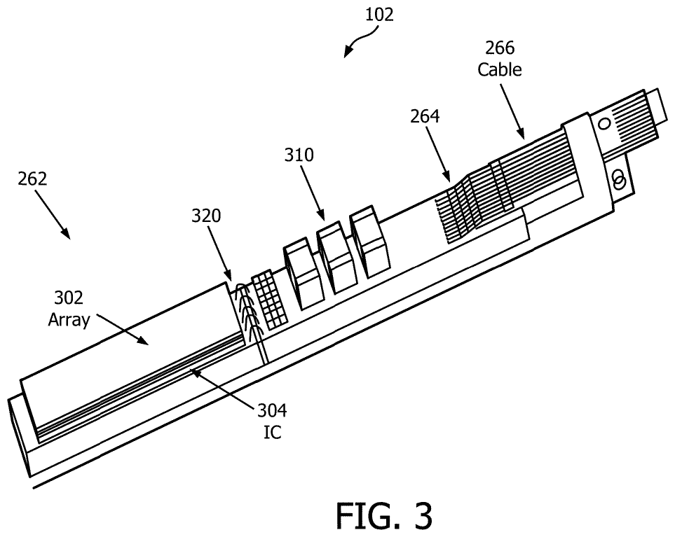

FIG. 3 is a top view of the imaging assembly 102 according to embodiments of the present disclosure. The imaging assembly 102 may include the imaging core 262 having an array of imaging elements 302 and micro-beam-former IC 304 that can be coupled to the array of imaging elements 302. The imaging assembly 102 also shows the electrical cable 266 coupled to the electrical interconnection 264. In some examples, the electrical cable 266 is further coupled through an interposer 310 to the micro-beam-former IC 304. In some examples the interposer 310 is connected to the micro-beam-former IC 304 through wire bonding 320.

In some embodiments, the array of imaging elements 302 is an array of ultrasound imaging transducers that are directly flip-chip mounted to the micro-beam-former IC 304. The transmitters and receivers of the ultrasound imaging transducers are on the micro-beam-former IC 304 and are directly attached to the transducers. In some examples, a mass termination of the acoustic elements is done at the micro-beam-former IC 304.

In some examples, the imaging assembly 102 includes an array of imaging elements 302 in the form of an array of more than 800 imaging elements and the electrical cable 266 includes a total of 12 signal lines or less. In some examples, the electrical cable 266 includes a total of 30 lines or less that includes the signal lines, power lines, and control lines. In some examples, an array of imaging elements, for example a one-dimensional or two-dimensional array, may include between 32 to 1000 imaging elements. For example, the array can include 32, 64, 128, 256, 512, 640, 768, or any other suitable number of imaging elements. For example, a one-dimensional array may have 32 imaging elements. A two-dimensional array may have 32, 64, or more imaging elements. In some examples, the number of signal lines are between 10 and 20, for example, 12 signal lines, 16 signal lines, or any other suitable number of signal lines. A one-dimensional array can be configured to generate two-dimensional images. A two-dimensional array can be configured to generate two-dimensional and/or three-dimensional images.

In some embodiments, the imaging assembly 102 includes an ultrasound transducer array with fewer than 30 wires connecting to the processing system 130. In certain embodiments, the 30 wires or less include 6-12 signal lines, preferably include 8 signal lines. In some examples, the transducer array is capable of two-dimensional and three-dimensional imaging. Additional aspects of the intraluminal imaging system includes a micro-beam-forming IC 304 with enough signal processing power to reduce the number of required ultrasound signal lines to a fraction of the total wires that include power and control lines.

In some examples, the electrical cable 266 of the imaging assembly 102 is directly coupled to the micro-beam-former IC 304 of the imaging assembly 102.

In some embodiments, the micro-beam-forming IC 304 lies directly underneath the array of acoustic elements 302 and is electrically connected to them. The array acoustic elements 302 may be piezoelectric or micromachined ultrasonic transducer (MUT) elements. In some examples, piezoelectric elements are attached to the IC 304 by flip-chip mounting of an assembly of acoustic layers that include sawing into individual elements. MUT elements may be flip-chip mounted as a unit or grown directly on top of the micro-beam-forming IC 304. In some examples, the cable bundle may be terminated directly to the micro-beam-forming IC 304, or may be terminated to an interposer 310 of suitable material such as a rigid or flexible printed circuit assembly. The interposer 310 may then be connected to the micro-beam-forming IC 304 via any suitable means such as wire bondings 320.

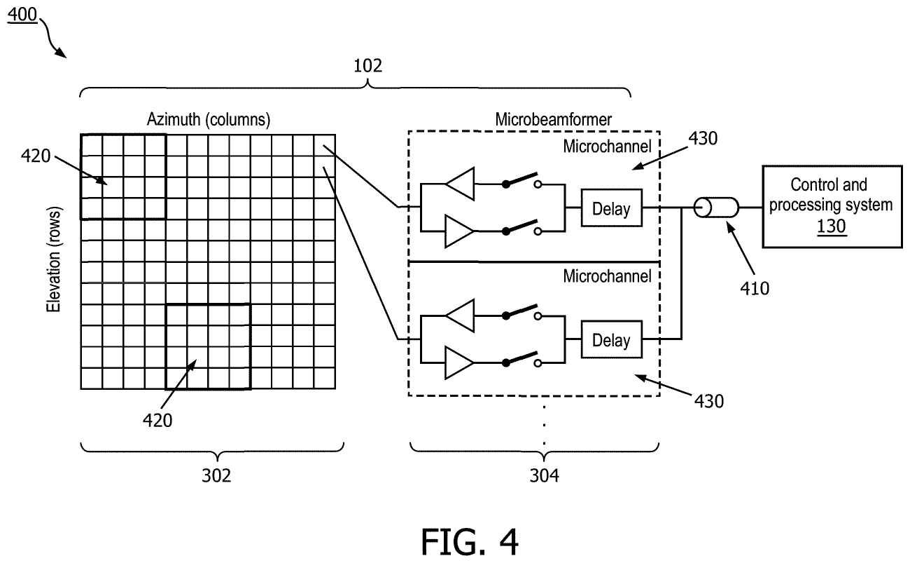

FIG. 4 is a schematic diagram 400 illustrating the beam-forming of an intraluminal imaging device according to embodiments of the present disclosure. The diagram 400 includes the imaging assembly 102 that includes the array of imaging elements 302 and micro-beam-former IC 304. The micro-beam-former IC 304 can be coupled to the array of imaging elements 302 at the distal portion of an ultrasound intraluminal imaging device (e.g., ultrasound imaging device 110). As shown, the array of imaging elements 302 is divided into one or more subarrays of imaging elements 420. For example, the array of imaging elements 302 are divided into 9 subarrays of imaging elements 420 that each has 16 imaging elements arranged as 4 by 4. The imaging assembly 102 also has the micro-beam-former IC 304 that includes a plurality of microchannels 430 that each can separately beam-form the signals received from imaging elements 420. As shown in FIG. 4, for example, the microchannels 430 each comprise a delay for alignment of the signals received from the imaging elements 420 of a subarray. As shown the microchannels delay lines 430 of each subarray of imaging elements 420 are separately coupled to one coaxial cable 410 such that the received signals of each subarray of imaging elements 420 are transferred through a separate channel, e.g., coaxial cable 410, to the control and processing system 130.

In some embodiments, the imaging assembly 102 includes an array of imaging elements 302. The array of imaging elements 302 can include two or more subarrays of imaging elements 420 of imaging elements. The imaging assembly 102 includes a micro-beam-former integrated circuit (IC) 304 coupled to the array of imaging elements.

In some examples, the micro-beam-former integrated circuit (IC) 304 can control the array of imaging elements 302 and can perform beam forming for a plurality of imaging elements of each subarrays of imaging elements 420 of the array of imaging elements 302.

In some embodiments, the imaging assembly 102 includes a cable 266 that includes two or more signal lines that are coupled to the micro-beam-former IC 304. Each of signal lines is associated with one of the subarrays of imaging elements 420 of the array of imaging elements 302 to transfer beam formed imaging signals of the associated subarray. For example, each signal line corresponds to an imaging element 420 and is configured to receive the beam-formed signals specific to the corresponding subarray.

In some embodiments, the electrical cable 266 further includes one or more power lines for feeding power to the micro-beam-former IC 304 and one or more control lines for communicating control signals to the micro-beam-former IC 304.

In some examples, imaging assembly 102 includes an array of imaging elements 302 in the form of an array of more than 800 imaging elements such that the array of imaging elements is divided into no more than 12 subarrays of imaging elements 420 and the cable 410 includes no more than 12 signal lines, each signal line associated with one subarray of imaging elements 420.

In some embodiments, the array of imaging elements 302 is a two dimensional array. In some examples, the array of imaging elements 302 is symmetric such that it has equal number of rows of imaging elements and columns of imaging elements. In some other examples, the array of imaging elements 302 is asymmetric such that it has different number of rows of imaging elements and columns of imaging elements.

In some embodiments, the micro-beam-former IC 304 includes multiple microchannel delay lines 430. The microchannel delay lines 430 are used to perform the beam forming for the plurality of imaging elements of each of the two or more subarrays of imaging elements 420. In some examples, the multiple microchannel delay lines 430 include at least one of a charge coupled device, an analog random access memory, or a tapped analog delay line.

In some examples, the first beam-formed signals and the second beam-formed signals are transmitted via a connection cable to a control and processing system 130 of FIGS. 1 and 4.

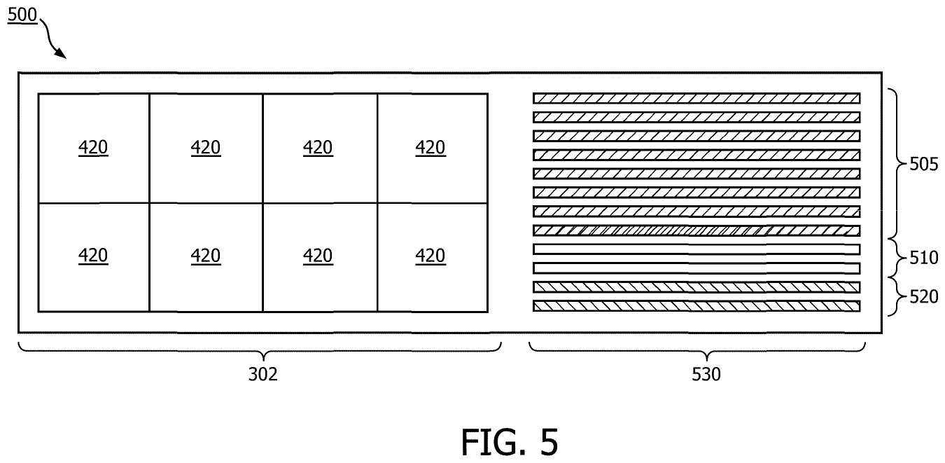

FIG. 5 is a schematic diagram illustrating aspects of an intraluminal imaging device according to embodiments of the present disclosure. The diagram 500 is consistent with the imaging assembly 102 of FIGS. 1-4 that includes the array of imaging elements 302 and micro-beam-former IC 304. As shown, the array of imaging elements 302 is divided into subarrays of imaging elements 420. For example, the array of imaging elements 302 is divided into eight subarrays of imaging elements 420. The diagram 500 also shows the cable 530 which is consistent with the cables 410 and 266 in FIGS. 3 and 4 and includes 8 signal lines 505, two control lines 510 and 2 power lines 520. As shown there are 8 subarrays of imaging elements 420 and one signal line for each subarray of imaging elements 420 such that each signal line is associated with on subarray of imaging elements 420 such that the received signals of each subarray of imaging elements 420 are transferred through a separate signal lines 505 that can be consistent with the coaxial cable 410 of FIG. 4 to the control and processing system 130. As shown the power lines 520/control lines 510 can be coupled to one or more subarray of imaging elements 420 and can provide power/control one or more subarray of imaging elements 420.

In some embodiments, as shown in FIGS. 4 and 5, the overall aperture is divided into subarrays of imaging elements 420 each of which is independently beam-formed. A two-dimensional array of imaging elements 302 is shown which can also be used for three-dimensional imaging. The essential element in the micro-beam-former IC 304 is the delay in each microchannel 430. The delay is used to time-align the echoes received by each element in the subarray of imaging elements 420 so that the signals add constructively in the desired beam direction, but destructively in other directions. The delay may be of any convenient sort of controlled variable delay, such as charge coupled devices (CCD's), analog RAM, tapped analog delay lines, etc. The amount of delay τ required depends on the size of the subarray of imaging elements 420 and the maximum steering angle θ:

τ=d sin θ/v

where d is the maximum dimension of the subarray and θ is the maximum beam steering angle, and v is the speed of sound in the object that is being imaged. In some examples, the area of a subarray of imaging elements 420 is proportional to the square of its dimension, so the maximum subarray area A is proportional to the square of the available delay:

A∝τ²

The larger the area of each individual subarrays of imaging elements 420, the fewer of them are needed to cover the entire acoustic aperture, the entire array of imaging elements 302. In some examples, each subarray of imaging elements 420 feeds one signal line through a single wire, thus, the number N of ultrasound signal wires required in the cable is inversely proportional to the square of the available delay:

N∝1/τ²

In some embodiments, the delay elements in use consist of a number of repeated elements, and the number of these elements determines the maximum available delay. Since the acoustic array is flip-chip mounted to the micro-beam-former IC 304, all of the processing, including the delay, for any given element can reside in the area occupied by that one element. In some examples, an ultrasound imaging catheter two-dimensional array may have 1000 or more elements, so the number of ultrasound signal wires required would be in the range of 30 to 50, and 15 to 20 power and control lines might also be needed. This number of wires is typical in existing one-dimensional ultrasound imaging catheters that use unshielded single wires rather than coaxial, and are individually attached to the acoustic elements. In some examples, the use of single wires rather than coax degrades the image due to noise susceptibility and crosstalk between the unshielded wires. In some examples, when using an IC within the catheter tip, the connections typically can be made to one narrow end of 2.5 mm, and so are limited to about 30 at most, including all of the ultrasonic signal lines, power lines, and control lines.

In some embodiments, newer IC processing equipment is now available which can approximately double the available amount of delay for the imaging signals, e.g., transducer signals. By the relations given above then the number of ultrasound signal wires could be reduced by about a factor of 4, to e.g., between 8 and 12. The total number of wires required is then in the range of 20 to 30, which is in the range of what that can be connected to the micro-beam-former IC 304, and allows the use of coaxial cables. In some examples, the reduced wire count has a number of advantages that include: having fewer interconnects in the flexible elongate member 108 tip, e.g., the catheter tip, decreases manufacturing cost and increases yield, and larger subarrays can track the depth of the received focal point in time.

In some examples, a possibly digital second beam-forming stage can be used that would further reduce the channel count. In some examples, cable count can further be reduced by implementing on-chip power regulation, sharing functions of wires, and using programmable autonomous IC controllers to reduce the number of power lines and control lines.

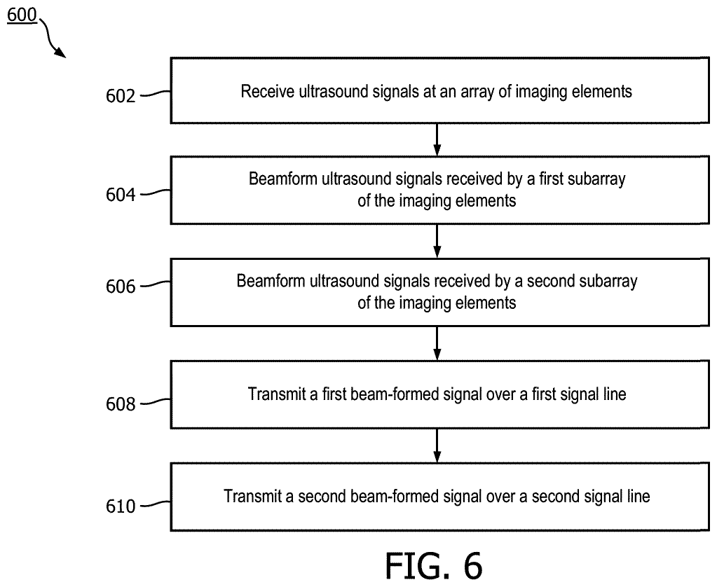

FIG. 6 provides a flow diagram illustrating a method 600 of intraluminal imaging of a vessel. As illustrated, the method 600 includes a number of enumerated steps, but embodiments of the method 600 may include additional steps before, after, and in between the enumerated steps. In some embodiments, one or more of the enumerated steps may be omitted, performed in a different order, or performed concurrently. The method 600 can be performed with reference to FIGS. 1, 2, 3, and 4. At step 602, ultrasound signals are received at an array of imaging elements, e.g., the array of imaging elements 302. The array of imaging elements 302 can be positioned within the distal portion 104 of an ultrasound imaging device 110. In some examples, a micro-beam-former IC 304 is directly coupled to the array of imaging elements 302 and transmits and receives imaging signals, e.g., ultrasound signals.

At step 604 of the method 600 the ultrasound signals received by the first subarray of the array of imaging elements 302 are beam-formed. The beam-forming can be performed with reference to FIGS. 3 and 4. In some embodiments, the micro-beam-former IC 304 is coupled, e.g., from beneath, to the array of imaging elements 302. The micro-beam-former IC 304 can command the array of imaging elements 302 and can transmit and receive signals, e.g., ultrasound signals. In some examples, the array of imaging elements 302 are divided into a plurality of subarrays of imaging elements 420 including the first subarray. The micro-beam-former IC 304 can also include a plurality microchannels delay lines 430. The micro-beam-former IC 304 can supply the required delays for beam-forming from one of the microchannels delay lines 430 to the first subarray to provide beam-forming for the first subarray such that the beam-forming is provided by applying the required delays to the signals of each of the plurality of the imaging elements of the first subarray. In some examples, the beam-forming is performed during both transmitting and receiving. In some other examples, the beam-forming is performed during the receiving. In some examples, the ultrasound signals received by the plurality of imaging elements of the first subarray of the array of imaging elements are beam-formed by applying the required delays to construct a first beam-formed signal.

At step 606 of the method 600 the ultrasound signals received by the second subarray of the array of imaging elements 302 are beam-formed. The beam-forming can be performed with reference to FIGS. 3 and 4. The micro-beam-former IC 304 can supply the required delays for beam-forming from one of the microchannels delay lines 430 to the second subarray to provide beam-forming for the second subarray such that the beam-forming is provided by applying the required delays to the signals of each of the plurality of the imaging elements of the second subarray. In some examples, the ultrasound signals received by the plurality of imaging elements of the second subarray of the array of imaging elements are beam-formed by applying the required delays to construct a second beam-formed signal.

At step 608 of the method 600, the first beam-formed signal is transmitted over a first signal line of a cable of the intraluminal imaging device. This step can be performed with reference to FIG. 4. The beam-formed signal is constructed by applying the required beam-forming delays provided by a microchannels delay line 430 of the micro-beam-former IC 304 to the received signals of the first subarray of imaging elements 420 and then transmitting a collection of the received and delayed signals of the first subarray of imaging elements 420 through the cable, e.g., coaxial cable 410 to the control and processing system 130.

At step 610 of the method 600, the second beam-formed signal is transmitted over a second signal line of a cable of the intraluminal imaging device. Likewise, this step can be performed with reference to FIG. 4. The beam-formed signal is constructed by applying the required beam-forming delays provided by a microchannels delay line 430 of the micro-beam-former IC 304 to the received signals of the second subarray of imaging elements 420 and then transmitting a collection of the received and delayed signals of the imaging elements 420 through a cable, e.g., a coaxial cable to the control and processing system 130. In some examples, the control and processing system 130 receives a plurality of the beam-formed signals from a plurality of the subarrays and constructs two-dimensional and three-dimensional images.

In some embodiments, the largest number of connections to a typical micro-beam-former IC 304 are the analog channel lines which carry the micro-beam-formed received signals back to the imaging system, and possibly transmit signals from the system 100 to the micro-beam-former IC. In some embodiments, large micro-beam-forming delays are produced on micro-beam-former IC 304 to reduce the number of analog channel lines compared to the existing micro-beam-former technology, thereby reducing the number of connections to the micro-beam-former IC 304 and the number of wires required to connect the imaging assembly 102 to the control and processing system 130. The reduced wire count has a number of advantages that include: reduced materials and assembly cost, reducing manufacturing cost and increasing yield, use of coaxial cables for transferring the signals and thus decreasing susceptibility to noise and crosstalk between channels that can degrade the image, ability to use larger wire size due to smaller number of wires and thus increasing reliability, providing three-dimensional imaging capability, simplifying interconnect from the cable to the micro-beam-forming IC, and providing the potential for automating the interconnect processes.

ICE transducers may use a phased array sensor comprising many small individual transducers, each with a separate wire connecting the catheter to the imaging console. Up to 128 wires may be needed, leading to high cost, difficult manufacturing, and compromised image quality. In phased array ICE transducers large number of wires can be brought up the catheter from the ICE transducer to the imaging system. A typical ICE transducer might have 128 transducers and 128 wires individually coupled to the transducers. These wires can all fit inside a catheter with a typical outer diameter of about 3 mm. The requirement to have so many wires in such a small diameter effectively precludes the use of coaxial cables for the wires as used in larger ultrasound imaging transducers. Without coaxial cables there is more crosstalk between signal channels and more interference from external noise sources, both of which will degrade the ultrasound image. Additionally, the wires can be individually connected to the elements of the transducer in a compact configuration to fit within the catheter tip. This difficult interconnect operation raises the cost of the transducer and is prone to errors and damage. Once assembled, the fine wires are prone to breaking due to flexure in normal use, decreasing the overall reliability of the transducer.

Another problem with the current art ICE transducers is that most of them create only two-dimensional images while clinicians would like to have the possibility of three-dimensional images. The only three-dimensional ICE transducer currently available has only a small field of view and compromised image quality. Micro-beam-forming is a technology that is used in larger ultrasound imaging transducers (e.g., Philips xMatrix, Clearvue, and Lumify transducer lines) both to create three-dimensional images and to reduce the number of wires required.

The demand for higher quality intraluminal images for ICE procedures requires the development of miniaturized imaging elements and catheter components. One of the challenges is to create an imaging assembly configured to fit into a catheter that is also capable of high-throughput processes, such as micro-beam-forming.

The present disclosure may offer solutions to these problems by providing an ultrasound assembly that includes an integrated circuit (IC) with a small number of channels. Particularly, the integrated circuit is configured to perform beam-forming processes, but is designed so that the number of wires required is less than for typical micro-beam-formed transducers. The reduction in wire count enables three-dimensional imaging, use of coaxial cable, higher manufacturing yield, reduced materials cost, and simpler, more easily manufactured electrical interconnect.

In some embodiments, the micro-beam-forming connections to the ultrasonic elements are simplified, e.g. by flip-chip mounting of the elements directly to the IC. This is advantageous for two-dimensional imaging transducers and nearly essential for three-dimensional imaging. Also, the number of wires required is reduced. Signal processing gains, especially for three-dimensional imaging come from having the micro-beam-former's transmitters and receivers directly attached to the transducer elements rather than at the end of a long cable. However, the IC requires digital control lines, electrical power, and a number of discrete capacitors for noise decoupling and energy storage for those power supplies. This creates a new interconnect problem to connect all of the signal wires, capacitors, and power supply lines to the IC. In larger micro-beam-formed transducers a combination of flexible and rigid printed circuits is typically used to connect to I/O pads along one or more edges of the IC. In an ICE transducer, the entire assembly may fit inside of the catheter tip which typically has a diameter of only 3 mm compared to the 2-5 cm diameter of the larger transducers. Additionally, due to the small diameter of the catheter, it is desirable to have the short dimension of the acoustic aperture fill the diameter as much as possible, so it is not desirable to use any of that dimensions (the long sides of the IC) for interconnect. This limits the interconnect to be at the ends of the IC which are short, typically no more than 2.5 mm. Additionally, it may be impossible to use both ends of the IC due to difficulties of routing the wires to both ends simultaneously. The limitation of using only one edge of less than 2.5 mm severely limits the number of connections that can be made. Due to size restrictions inside the catheter, probably only one row of I/O pads could be connected along that edge, so a maximum of about 30 connections could be made with modern bonding equipment. Practical considerations related to processing the catheter can force even a smaller number.

Embodiments of the present disclosure, such as the beam-forming applications of the present disclosure, may include features similar to those described in U.S. Provisional App. No. 62/403,479, filed Oct. 3, 2016, U.S. Provisional App. No. 62/434,517, filed Dec. 15, 2016, U.S. Provisional App. No. 62/403,311, filed Oct. 3, 2016, U.S. Provisional App. No. 62/437,778, filed Dec. 22, 2016, U.S. Provisional App. No. 62/401,464, filed Oct. 29, 2016, U.S. Provisional App. No. 62/401,686, filed Oct. 29, 2016, and/or U.S. Provisional App. No. 62/401,525, filed Oct. 29, 2017, the entireties of which are hereby incorporated by reference herein.

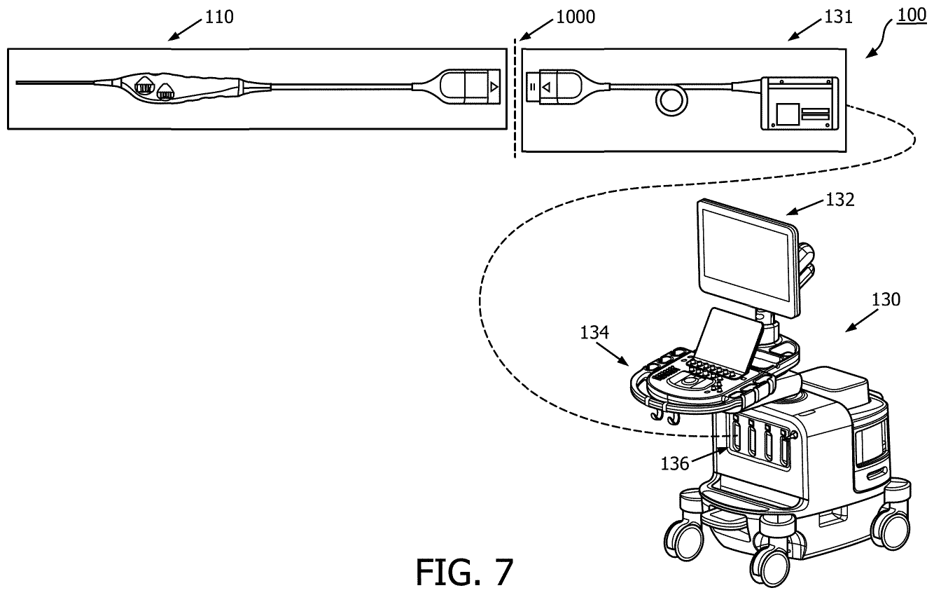

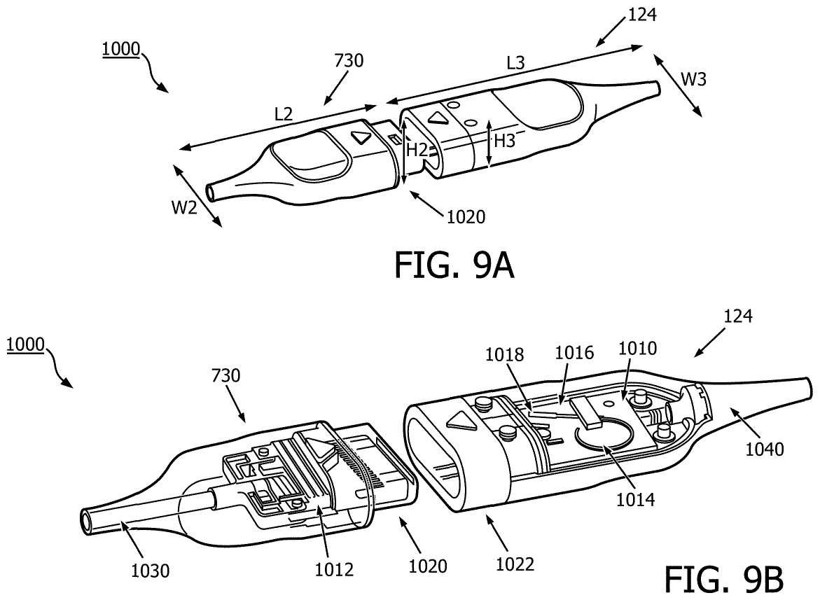

FIG. 7 is a schematic diagram of an ultrasound imaging system 100 according to embodiments of the present disclosure. One or more components of the system 100 of FIG. 7 can include features similar to those shown and described with respect to FIGS. 1-6. The system 100 may include the ultrasound imaging device 110, the control and processing system 130, and a patient interface module (PIM) 131 extending between the device 110 and the processing system 130. For example, the PIM 131 may provide a physical and electrical connection between the ultrasound imaging device 110 and the control and processing system 130. Some embodiments of the present disclosure omit the PIM 131. In other embodiments, the PIM 131 is communicatively interposed between the ultrasound imaging device 110 and the processing system 130. In some instances, the PIM 131 can be referenced as a patient interface cable. For example, a proximal connector of the ultrasound imaging device 110, a distal connector of the PIM, and/or a proximal connector of the PIM may be configured to couple the ultrasound imaging device 110, the PIM 131, and the control and processing system together mechanically and electrically. The system 100 may include may include a connector assembly 1000 comprising a connector of the ultrasound imaging device 110 and the PIM 131 that is discussed in more detail in reference to FIGS. 9A and 9B.

In some embodiments, the control and processing system 130 may include one or more computers, processors, and/or computer systems. The control and processing system 130 may also be referred to as a console. In some embodiments, the PIM 131 is in mechanical and electrical communication with the control and processing system 130, such that the electrical signals are transmitted the ultrasound imaging device 110 through the PIM 131 and to the control and processing system 130. The control and processing system 130 may include one or more processors and/or memory modules forming a processing circuit that may process the electrical signals and output a graphical representation of the imaging data on the monitor 132. One or more electrical conductors of the ultrasound imaging device 110 and PIM 131 may facilitate communication between the control and processing system 130 and the ultrasound imaging device 110. For example, a user of the control and processing system 130 may control imaging using the ultrasound imaging device 110 via a control interface 134 of the control and processing system 130. Electrical signals representative of commands from the control and processing system 130 may be transmitted to the ultrasound imaging device 110 via connectors and/or cables in the PIM 131 and the ultrasound imaging device 110. The control and processing system 130 may be transportable and may include wheels or other devices to facilitate easy transportation by a user.

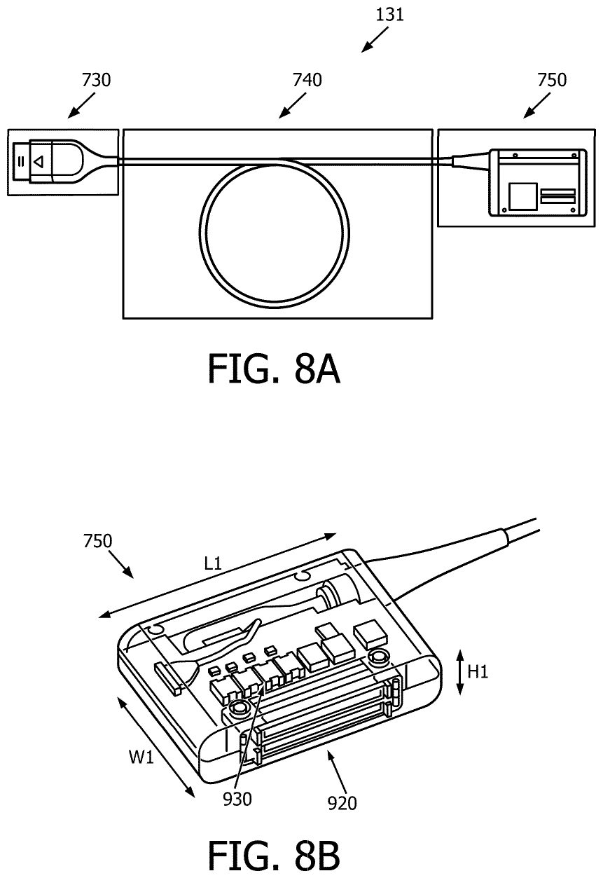

In some embodiments, the PIM 131 includes a distal connector 730 and a proximal connector 750 (as shown in, e.g., FIG. 8A) that are removably connectable to the ultrasound imaging device 110 and control and processing system 130, respectively. In particular, the ultrasound imaging device 110 and PIM 131 may be connected at a connector assembly 1000 which is discussed in more detail in reference to FIGS. 9A and 9B. In some embodiments, the one or more components of the ultrasound imaging device 110 may be disposable components. For example, a user, such as a physician, may obtain the ultrasound imaging device 110 in a sterilized packaging. In some embodiments, the ultrasound imaging device 110 may be disposed after a single use. In other embodiments, the ultrasound imaging device 110 can be sterilized and/or re-processed for more than one use. The PIM 131 may be a re-usable component that is used in multiple procedures. For example, the PIM 131 can be cleaned between individual procedures, such as being treated with disinfectants to kill bacteria. In some embodiments, the PIM 131 may not be required to be sterilized before a medical procedure. For example, the PIM 131 can be sufficiently spaced from the patient such that use of a non-sterile PIM 131 is safe for the patient. The sterile-nonsterile connection at the connector assembly 1000 between the ultrasound imaging device 110 and the PIM 131 may allow for a safe operating environment while saving costs by allowing expensing equipment to be reused.

While some embodiments of the present disclosure refer to an imaging device, an ultrasound imaging device, or an intraluminal imaging device, it is understood that the ultrasound imaging device 110 and the system 100 generally can be used to image vessels, structures, lumens, and/or any suitable anatomy/tissue within a body of a patient including any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the imaging device 110 may be may be used to examine man-made structures such as, but without limitation, heart valves, stents, shunts, filters and other devices. For example, the ultrasound imaging device 110 can be positioned within fluid filled or surrounded structures, both natural and man-made, such as within a body of a patient. The vessels, structures, lumens, and anatomy/tissue can include a blood vessel, as an artery or a vein of a patient's vascular system, including cardiac vasculature, peripheral vasculature, neural vasculature, renal vasculature, and/or any suitable lumen inside the body.

FIG. 8A is an illustration of the patient interface module (PIM) 131, according to aspects of the present disclosure. The PIM 131 includes a proximal connector 750, a distal connector 730, and a cable 740 extending between the proximal connector 750 and the distal connector 730. The distal connector 730 is configured to be mechanically and/or electrically coupled to the imaging device 110. The proximal connector 750 is configured to be mechanically and/or electrically couple to the control and processing system 130. For example, the proximal connector 750 is configured to be inserted into one a slot 136 on the control and processing system 130 (FIG. 7). The proximal connector 750 and the slots 136 may include any suitable connections that are configured to mechanically and/or electrically couple to one another. In some embodiments, the conduit or cable 740 can be referenced as a flexible elongate member. The cable 740 includes one or more electrical conductors to facilitate electrical communication between the proximal connector 750 and the distal connector 730. As a result, the PIM 131 facilitates electrical communication between the ultrasound imaging device 110 and the processing system 130.

FIG. 8B is a perspective view of the proximal connector 750 of the PIM 131, according to embodiments of the present disclosure. The proximal connector 750 can include one more electronic component to facilitate communication with the processing system 130. For example, the proximal connector 750 includes a connector module 920. The connector module 920 may be disposed on a side surface of the proximal connector 750. In some embodiments, the connector module 920 can include one or more male or female zero insertion force (ZIF) connectors. In such embodiments, the slots 136 of the processing system 130 may include corresponding female or male ZIF connectors. As such, when the proximal connector 750 of the PIM 131 is inserted into the one or more slots 136 (FIG. 7), the male/female connectors in the PIM 131 are electrically connected to the female/male connectors in the slots 136. Generally, the connector module 920 can be any suitable type of male or female electrical connector. For example, electrical connectors can include one or more types of connectors, such as low insertion force (LIF) connectors, flat flexible connectors (FFC), ribbon cable connectors, and serial advanced technology attachment (SATA) connectors.

In some embodiments, the proximal connector 750 includes a number of electrical transformers 930. These transformers 930 facilitate electrical isolation of the high voltage signals associated with the imaging assembly 102 of the ultrasound imaging device 110. In that regard, the patient safety is facilitated by providing electrical isolation. At the same time, a minimal number of transformers 930 can provide such safety while being positioned in the relatively smaller and less heavy body of the proximal connector 750. In some embodiments, the number of transformers 930 in the proximal connector 750 is equal to the number of data channels output by the ultrasound imaging device 110. For example, there may be 8 data channels and 8 transformers 930 in the proximal connector 750. In other embodiments, there may be 10 data channels and transformers, 6 data channels and transformers, or other suitable numbers of channels and transformers.

Advantageously, the PIM 131 is configured with features to avoid the bulky components of existing systems (as described, e.g., with respect FIGS. 13 and 14). For example, the ultrasound imaging device 110 may include one or more Application Specific Integrated Circuits (ASICs) which may be configured to provide signal generation (whereas existing systems generated signals at the console and transmitted the signal to the imaging device). The ASICs may also provide beam-forming such that the number of distinct channels or communication lines along the cable 740 is reduced. For example, the ASICs of the ultrasound imaging system 100 may sum about 850 channels and reduce this number to about 8 channels. Prior art systems needed a PIM with completely separate isolation box to house large number of isolation transformers, e.g., sixty-four transformers because of the larger number of channel/communication lines. The ultrasound imaging device 110 allows for the proximal connector 750 of the PIM 131 to include, e.g., eight transformers in a relatively small and low-weight package.

FIGS. 9A and 9B are perspective views of a connector assembly 1000 including a proximal connector 124 of the ultrasound imaging device 110 and the distal connector 730 of the PIM 131, according to embodiments of the present disclosure. FIG. 9B illustrates exemplary interior and exterior components of the connector assembly 1000. The proximal connector 124 and the distal connector 730 can be connected to establish mechanical and/or electrical communication between the ultrasound imaging device 110 and the PIM 131. In the illustrated embodiment, the distal connector 730 includes a male type connector module 1020 while the connector 124 includes a female type connector module 1022. In other embodiments, the connector 124 includes a male type connector and the connector 730 includes a female type connector.

Referring to FIGS. 8A, 8B, 9A, and 9B, generally, the connectors of the PIM 131 (e.g., the proximal connector 750 and the distal connector 730) and the proximal connector 124 of the ultrasound imaging device 110 may include any suitable connections that are configured to mechanically and/or electrically couple to other connections or devices. In some embodiments, the connectors 124, 730, and 750 may also be configured to be water resistant or waterproof, such as being designed for an IPX4 rating, as well as other ratings.

One or more of the connectors 124, 730, and 750 can include various electronic components to facilitate transmission of signals between the ultrasound imaging device 110 and the control and processing system 130. In some embodiments, the distal connector 730, the proximal connector 750, and the connector 124 are configured to include one or more PCB boards. These PCB boards may include one or more electronic components, including memory, buffers, amplifiers, other integrated circuits, and other components. For example, the distal connector 730 and the proximal connector 750 may include one or more memories to store data, for example, a cache memory (e.g., a cache memory of the processor), read-only memory (ROM), programmable read-only memory (PROM), erasable programmable read only memory (EPROM), electrically erasable programmable read only memory (EEPROM), a solid state memory device, or other forms of volatile and non-volatile memory. In some embodiments, one or more of the distal connector 730 and the proximal connector 750 include an EEPROM that allows the ultrasound imaging device 110 to be able to interact in real time with the control and processing system 130. For example, a memory device of the proximal connector 124 of the imaging device 110 can store identifying information about the imaging assembly 102, such as the frequency of the ultrasound transducers, the date of manufacture, the date of first/last use, the number of permissible uses, etc. The identifying information can be queried by the processing system 130, and the system 130 can control operation of the ultrasound imaging device 110 based on the identifying information, including allowing operation of the imaging assembly 102, processing the ultrasound imaging data obtained by the imaging assembly 102, etc.

The connectors 124, 730, and/or 750 may also include one or more integrated circuits configured to increase the stability and/or integrity of signals transmitted from the ultrasound imaging device. These integrated circuits may include one or more buffers, including an array of buffers. The connectors 124, 730, and/or 750 may also include one or more amplifiers to strengthen signals transmitted from the ultrasound imaging device to provide for better image quality. In some embodiments, the connectors 124, 730, and/or 750 are long and very small in diameter, which may cause a loss in signal amplitude when signals traverse the connectors 124, 730, and/or 750. An amplifier integrated circuit may be inserted into each connector 124, 730, and/or 750 to restore the amplitude of signals. For digital signals passing through the connectors 730, 124 to the imaging assembly 102, signal amplitude levels may be restored with digital buffer amplifiers inserted into each digital line in the PIM 131. Furthermore, the connectors 124, 730, and/or 750 may include PCBs for other design parameters. For example, conductive wires extending from the imaging assembly 102 of the ultrasound imaging device 110 may terminate directly onto a PCB within proximal connector 124.

In some embodiments, the connector assembly 1000 includes a connector 124 of the ultrasound imaging device 110 that is configured to connect to a distal connector 730 of the PIM 131. The connectors 124, 730 may include one or more PCB boards 1010, 1012 with electronic components, as well as connector modules 1020, 1022. The PCB boards 1010, 1012 may include one or more substrates (such as substrate 1016), flexible PCB boards, connectors, cables, and other components. The connector modules 1020, 1022 may include a number of pins or other electrical connections that are operable to provide electrical connections between the connectors 124, 730. The PCB boards 1010, 1012 within the connectors 124, 730 may be electrically connected through the connector modules 1020, 1022 such that data may be transmitted through the connector assembly 1000 when the connector modules 1020, 1022 are in contact.

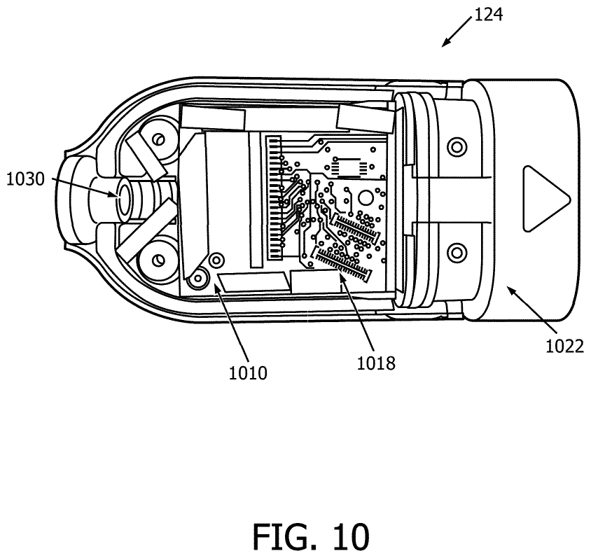

In some embodiments, connector 124 may include a first PCB board 1010 as well as a substrate 1016 configured to serve as a termination point for a number of wires 1014 extending through the ultrasound imaging device 110. In some embodiments, the wires 1014 may be similar to the electrical cables 266 discussed in reference to FIG. 3, and may extend from an imaging device through the ultrasound imaging device 110. In some embodiments, the substrate 1016 is attached to the PCB board 1010 at a port 1018, as further shown in FIG. 10. The wires 1014 may be routed through the ultrasound imaging device, such as bundled together or through one or more twists or loops, such as the loop shown in FIG. 9B. The shape and size of the wires 1014 may allow the ultrasound imaging device to be articulable while allowing for the transfer of large amounts of imaging data. The connectors 124, 730 may also include cables 1030, 1040 extending out from the connectors 124, 730. The cables 1030, 1040 may serve as anchor points for the PCB boards 1010, 1012 as well as providing stability for electrical wires or connections extending through the ultrasound imaging device 110 and the PIM 131.

With reference to FIGS. 8B, 9A, and 9B, the proximal connector 750 of the PIM 131, the distal connector 730 of the PIM 131, and the proximal connector 124 of the imaging device 110 advantageously have a relatively smaller profile and relatively lighter weight than prior art devices. Accordingly, a user in a clinical environment is advantageously able to connect the ultrasound imaging device 110, PIM 131, and/or processing system 130 using connectors 124, 730, and 750 that are easier to handle because they are smaller and lighter than prior art devices. In some embodiments, the proximal connector 750 of the PIM 131 weighs approximately 275 grams or 0.6 lbs. In other embodiments, the proximal connector 750 of the PIM 131 weighs approximately 0.8 lbs, between 0.5 lbs and 0.6 lbs, between 0.7 and 0.9 lbs, between 0.75 lbs and 1 lb, or between 0.5 and 1.2 lbs. A connector module 1020 may be included on a distal end of the distal connector 730. In some embodiments, the connector module 1020 is smaller than the connector module 920. In some embodiments, the distal connector 730 of the PIM 131 weighs approximately 95 grams or 0.2 lbs, between 0.1 and 0.3 lbs, between 0.15 lbs and 0.25 lbs, or between 0.05 and 0.3 lbs. In some embodiments, the combined weight of the PIM 131 is approximately 460 grams or 1 lb. In other embodiments, the combined weight of the PIM 131 is less than 1 lb, about 1 lb, between 0.8 and 1.2 lbs, or between 0.75 and 1.25 lbs.

In some embodiments, the proximal connector 750 has a length L1 of approximately 4.36 inches, a width W1 of approximately 3 inches, and a height H1 of approximately 1.1 inches. In other embodiments, length L1 may be between 4 and 4.5 inches, between 3 and 5 inches, or between 4.3 and 4.4 inches, as well as other lengths. The width W1 may be between 2 and 4 inches, between 1 and 5 inches, or between 2.5 and 3.5 inches, as well as other widths. The height H1 may be between 1 and 2 inches, between 0.5 and 1.5 inches, or between 1.1 and 1.2 inches, as well as other heights.

In some embodiments, the distal connector 730 has a length L2 of approximately 4.75 inches, a width W2 of approximately 2 inches, and a height H2 of approximately 1 inch. In other embodiments, length L2 may be between 4.5 and 5 inches, between 4.6 and 4.9 inches, or between 4.7 and 4.8 inches, as well as other lengths. The width W2 may be between 1.9 and 2.1 inches, between 1.75 and 2.25 inches, or between 1.9 and 2.1 inches, as well as other widths. The height H2 may be between 0.75 and 1.25 inches, between 0.95 and 1.05 inches, or between 0.9 and 1.1 inches, as well as other heights.

In some embodiments, the connector 124 has a length L3 of approximately 4 inches, a width W3 of approximately 2 inches, and a height H3 of approximately 1 inch. In other embodiments, length L3 may be between 3.9 and 4.1 inches, between 3.75 and 4.25 inches, or between 3.95 and 4.05 inches, as well as other lengths. The width W3 may be between 1.75 and 2.25 inches, between 1.9 and 2.1 inches, or between 1.95 and 2.05 inches, as well as other widths. The height H3 may be between 0.75 and 1.25 inches, between 0.95 and 1.05 inches, or between 0.9 and 1.1 inches, as well as other heights.

FIG. 10 is an overhead perspective view of a connector 124 of the ultrasound imaging device 110, showing the PCB board 1010, connector module 1022, and strain relief or cable inlet 1040. The PCB board 1010 may include a number of electrical components, including integrated circuits and vias. The PCB board 1010 may include one or more ports 1118 that may provide a connection to other substrates, such as substrate 1016 shown in FIGS. 9, 11, and 12. The PCB board 1010 may include one or more memory modules, amplifiers, buffers, or other integrated circuits.

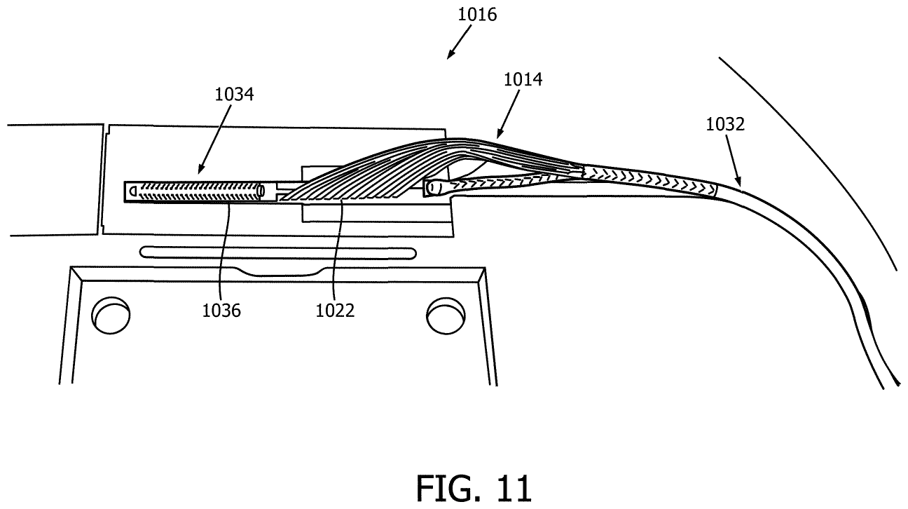

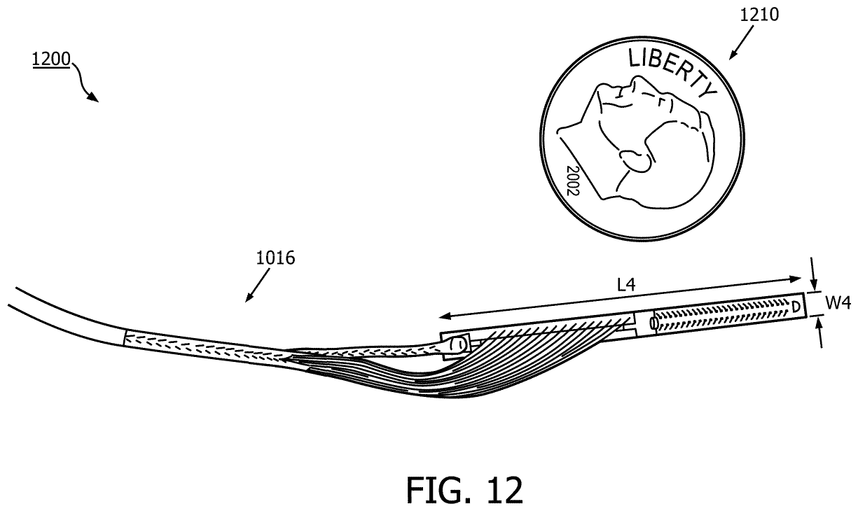

FIG. 11 is a perspective view of the substrate 1016 and wires 1014. The wires 1014 may be bundled into a cable 1032 as shown in the example of FIG. 11. Furthermore, the substrate 1016 may include a connector portion 1034 including a number of electrical connectors 1036. These electrical connectors 1036 may connect to other connectors within the port 1018 as shown in FIGS. 9 and 10. The substrate 1016 may serve as a termination point for the wires 1014 and may include converters (such as analog-to-digital or digital-to-analog converters) to transfer the data from the wires 1014 to other electrical components, including various integrated circuits. In some embodiments, the substrate 1016 (FIGS. 11 and 12) may be a flexible substrate, while the PCB 1010 (FIG. 10) and/or other PCBs are rigid substrates.

FIG. 12 is a comparison 1200 of an exemplary substrate 1016 and a dime 1210 to show the approximate size of the substrate 1016. In some embodiments, the substrate 1016 may have a length L4 of approximately 1.2 inches. The length L4 may be between 1 and 1.25 inches, between 0.5 and 1.5 inches, or between 1.2 and 1.3 inches, as well as other lengths. The substrate 1016 may have a width W4 of approximately 0.08 inches. The width W4 may be between 0.05 and 0.10 inches, between 0.075 and 0.085 inches, or between 0.07 and 0.09 inches, as well as other widths. The small size of the substrate 1016 and fine wires 1014 may provide for a smaller PCB board within the connector 124 of the ultrasound imaging device 110.

As described above, FIG. 13 illustrates a prior art patient interface module (PIM) that is large, heavy, and can be cumbersome to use. For example, as illustrated in FIG. 14, the large connector 1350 of an imaging device is connected to the isolation box 1310. In contrast, embodiments, of the present disclosure provide a PIM 131, and connectors 124, 730, and/or 750 that are smaller, lighter, and easier to use. For example, the PIM 131 does not require a distinct isolation box 1310 because electrical isolation transformers can be provided in proximal connector 750 of the PIM 131. Because the transformers are disposed in the proximal connector 750 of the PIM 131, the connector assembly 1000 between the PIM 131 and the ultrasound imaging device 110 (including the connectors 124 and 730) can provide a much smaller footprint by including smaller PCB boards and significant weight savings (i.e., two or more lbs.). As shown in the comparison of FIG. 14, the connector assembly 1000 provides a much smaller cross section, weighs less, and is more easily transported in a medical environment. This may also allow for easier storage of the connector. For example, the PIM 131 may be stored within the processing system 130, such as in a bay beneath the slots 136. The reduced size and weight of the connector assembly may also allow the addition of accessories to the connector assembly 1000 that would not be possible with the existing obtrusive and heavy PIM shown in FIG. 13. For example, bed rail hooks, EPIQ holders, bedside interfaces, and other accessories may be added to the PIM. Furthermore, the connector assembly 1000 may provide substantial cost and time savings by including a disposable portion (connector 124) and a non-disposable portion (distal connector 730). Additionally, PIM 131 may provide improved flexibility of design over existing PIMs. For example, by incorporating PCBAs inside each connector of the connector assembly 1000, a designer may be able to redesign the circuitry, add/remove current electrical designs/components, and/or adapt connectors to other devices so long as the required electrical changes can fit on the defined board. In this way, the same connectors can be used across various situations or projects by only changing the internal PCBA in the connectors. Thus, the connectors within the connector assembly are more adaptable than existing connectors.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging system, comprising:
a patient interface module communicatively positioned between an ultrasound imaging device and a console, the patient interface module comprising:
a proximal connector configured to interface with the console;
a distal connector configured to interface with a connector of the ultrasound imaging device; and
a cable extending between the proximal connector and the distal connector,
wherein the proximal connector comprises a plurality of transformers.

2. The system of claim 1, further comprising the ultrasound imaging device.

3. The system of claim 2, wherein the ultrasound imaging device comprises an intra-cardiac echocardiography (ICE) catheter.

4. The system of claim 2, wherein the ultrasound imaging device is configured to output signals via a plurality of channels, wherein a quantity of the plurality of transformers equals a quantity of the plurality of channels.

5. The system of claim 4, further comprising the console, wherein the console is configured to process the signals output by the ultrasound imaging device and display an ultrasound image based on the processed signals.

6. The system of claim 1, wherein the distal connector has a width of approximately 2 inches and a length of approximately 4.75 inches.

7. The system of claim 1, wherein the proximal connector has a width of approximately 3 inches and a length of approximately 4.36 inches.

8. The system of claim 7, wherein the distal connector has a weight of approximately 0.2 lbs. and the proximal connector has a weight of approximately 0.6 lbs.

9. The system of claim 1, wherein the distal connector comprises one or more electronic components.

10. The system of claim 9, wherein the one or more electronic components comprise a memory configured to facilitate communications between the ultrasound imaging device and the console.

11. The system of claim 9, wherein the one or more electronic components comprise a plurality of integrated circuits configured to increase signal integrity.

12. The system of claim 9, wherein the one or more electronic components comprise a plurality of amplifiers configured to strengthen signals output by the ultrasound imaging device.

13. An ultrasound imaging system, comprising:
an ultrasound imaging device in communication with a console, comprising:
a flexible elongate member comprising a proximal portion and a distal portion;
an ultrasound transducer at the distal portion;
a plurality of conductive wires extending along the flexible elongate member; and
a connector at the proximal portion, wherein the connector comprises:
a printed circuit board assembly (PCBA) configured to transmit imaging data obtained by the ultrasound transducer.

14. The system of claim 13, wherein the plurality of conductive wires extends from the ultrasound transducer to the connector and provides an electrical connection between the ultrasound transducer and the connector.

15. The system of claim 13, wherein the connector comprises a first substrate at which the plurality of conductive wires terminates and a second substrate in communication with the first substrate, wherein the PCBA is disposed on the second substrate.

16. The system of claim 15, wherein the first substrate is attached to a coupling module on the second substrate.

17. The system of claim 13, wherein the proximal connector has a width of approximately 3 inches and a length of approximately 4.36 inches.

18. The system of claim 15, wherein the first substrate has a width of approximately 0.01 inch and a length of approximately 0.5 inch.

19. The system of claim 13, wherein each conductive wire serves as a data channel to transmit data from the ultrasound transducer to the connector.

20. The system of claim 13, wherein the imaging data is stored in an EEPROM within the PCBA.
